(19) United States
(12) Patent Application Publication
(10) Pub. No.: US 2026/0141522 A1
(43) Pub. Date: May 21, 2026

(54) SYSTEMS AND METHODS FOR GENERATIVE, PHYSICS-INFORMED MEDICAL IMAGES SYNTHESIS OR TRANSLATION

(71) Applicant: Ebenbuild GmbH, Munich (DE)

(72) Inventors: Jonas BIEHLER, Grafing b. Munich (DE); Giorgia CARADONNA, Munich (DE)

(21) Appl. No.: 19/388,102

(22) Filed: Nov. 13, 2025

Related U.S. Application Data
(60) Provisional application No. 63/720,977, filed on Nov. 15, 2024.

(51) Int. Cl. G06T 7/00; A61M 16/00

(52) U.S. Cl. CPC G06T 7/0014; A61M 16/024; G06T 2207/10081; G06T 2207/10088; G06T 2207/10108; G06T 2207/20081; G06T 2207/20084; G06T 2207/30061

(57) ABSTRACT

Systems and methods for generating synthetic computed tomography (CT) images, such as generating synthetic time-resolved four-dimensional CT or single-photon emission CT images of the human respiratory system, are described. Generating a synthetic CT image includes processing at least one output from a physics-based lung simulation model in combination with at least one generative learning model. The output from the physics-based lung simulation model provides additional information that improves clinical decision-making without additional patient exposure to radiation-intensive imaging procedures.

### CROSS REFERENCE TO RELATED APPLICATIONS

This application claims priority to U.S. Provisional Application No. 63/720,977, filed on Nov. 15, 2024, the entire disclosure of which is hereby incorporated by reference in its entirety.

### TECHNICAL FIELD

Aspects of the present disclosure relate generally to devices, systems and related methods for medical imaging. Specifically, the disclosure relates to systems and methods for generating synthetic computed tomography images of anatomy, among other aspects.

### BACKGROUND

Advancements in medical imaging, such as computed tomography (CT), allow a medical professional to scan a volume of tissue and to generate a three-dimensional (3D) representation of the scanned volume. Four-dimensional computed tomography (4D CT) and single-photon emission computed tomography (SPECT) are two imaging modalities commonly used during a medical procedure. 4D CT captures a series of 3D images of a subject over a period of time, allowing visualization of dynamic processes within the subject, such as respiratory motion. SPECT visualizes processes within a subject by detecting gamma rays emitted by radioactive tracers, such as a radiolabeled aerosol, which may be inhaled by the subject, and which emits detectable gamma rays as the radioactive tracer decays. However, it would be useful to mitigate the subject's exposure to radiation (e.g., gamma rays, x-rays) associated with CT medical imaging procedures (e.g., SPECT, 4D CT). Embodiments of the disclosure discussed herein address this issue, among others, in the field of medical imaging devices, systems and related methods.

The foregoing general description and the following detailed description are exemplary and explanatory only and are not restrictive of the disclosure. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

According to one aspect, the present disclosure provides a machine-implemented method comprising: receiving at least a first image of at least a portion of an organ, the first image obtained using a first imaging modality. A computational model may be determined that simulates and/or predicts, using the first image, one or more functional, mechanical, chemical, biological, and/or physiological quantities across a domain of at least the portion of the organ. An output of the computational model may be provided to a generative model, the generative model comprising one or more of generative adversarial network(s) (GANs), diffusion model(s), and/or other generative model(s) to: (i) receive and/or generate a second image of the organ obtained using a second imaging modality different from the first imaging modality, or (ii) generate a second image of the organ associated with the first imaging modality under different real or simulated conditions than the first image, the different real or simulated conditions comprising different time, different mechanical, or different physiological conditions, or generate a combination of both (i) and (ii).

According to some aspects, the organ may include the lungs or the respiratory system. The organ may also comprise the heart or cardiovascular system, the brain, the liver and other abdominal organs, the musculoskeletal system, the vascular system, or pathologies such as tumors. The computational model output may be used to condition a generative model that was trained on a number of samples, such that the samples include data in which either the image taken with the different imaging modality may be available or the organ was imaged with the same modality at another time or under other conditions.

### BRIEF DESCRIPTION OF DRAWINGS

FIG. 1A depicts an example process for generating synthetic images of an organ.

FIG. 1B depicts an example process for training a generative model for use in the example process of FIG. 1A.

FIG. 1C shows a schematic illustration of one possible embodiment of the disclosure, in particular the use of a generative model to obtain four-dimensional Computed Tomography (4D CT) images.

FIG. 1D shows a schematic illustration of one possible embodiment of the disclosure, in particular the use of a generative model to determine Single-Photon Emission Computed Tomography/Computed Tomography (SPECT/CT) images.

FIG. 2A details the use of a generative model to obtain 4D CT images of the lungs used in one variant of the embodiment in FIG. 1C.

FIG. 2B details the use of a generative model to obtain displacement vector fields for the lung images used in another variant of the embodiment in FIG. 1C.

FIG. 3 details the use of preferred functional blocks of an architecture for a vox2vox model, as used in the embodiments in FIGS. 1C-1D.

FIG. 4 shows a schematic illustration of a possible architecture for a generator of the vox2vox model of the embodiments in FIGS. 1C-1D.

FIG. 5 shows a schematic illustration of a possible discriminator architecture for the vox2vox model of the embodiments in FIGS. 1C-1D.

FIG. 6 shows a schematic representation of a possible ResNet block for an architecture of a reverse diffusion model, as used in the embodiment in FIG. 1D.

FIG. 7 shows a schematic representation of a possible architecture for a reverse diffusion model based on a U-Net architecture for the embodiment in FIG. 1D.

FIG. 8 shows an example computer.

### DETAILED DESCRIPTION

As discussed herein, physics-based simulation models may be used to determine patient-specific fields such as strain or particle deposition data from medical images. A generative model may use the medical image and/or the physics-based simulation output to generate new images, including synthetic 4D CT data and/or synthetic SPECT images.

There are multiple techniques that may be used to synthesize 4D CT data: for example, a generative model that directly synthesizes data, or one that generates displacement vector fields (DVF) used to warp input 3D CT scans.

Another technique based on neural networks is training conditional generative adversarial networks (cGANs) on 4D CT images. For example, 4D respiratory motion synchronized image synthesis may be performed from static CT images using the cGANs. The cGANs may be used to run several independent image-to-image translation networks in parallel, where each network synthesizes one respiratory state of the 4D CT data and may have a pix2pix architecture. However, a limitation of this approach is that the synthesized respiratory states represent typical average patterns rather than reflecting the actual patient-specific motion. To generate realistic respiratory dynamics, the cGAN may be doubly conditioned on the 3D CT image and a scalar value representing the respiratory state, e.g., the lung volume. For example, a 3D vox2vox architecture may be used for the cGAN. One further alternative consists in generating the DVFs of the respiratory cycle and then warping the 3D CT image. CT respiratory motion synthesis may be performed using joint supervised and adversarial learning. The DVF may be the vector field of the displacement vectors for all corners of the voxels. This method may employ an adversarial term jointly with the magnitude of the DVF and the warped image to circumvent excessive smoothness typically obtained by conventional approaches.

While the methods discussed above allow a conditioning through a global scalar value, techniques discussed herein below may rely on regional lung information, in the form of the output of a lung simulation model, to condition the model as part of the input. Regional lung information helps to inform the heterogeneity that a pathological lung presents. Therefore, techniques described herein can provide conditioning based on more extensive information on the respiratory system, which results in more accurately generated images and requires less training data as the algorithm does not have to infer all information about lung physiology, pathology, and function from images alone.

Concerning the generation of SPECT or single-photon emission computed tomography/CT fusion images (SPECT/CT) images, one approach includes generating, from real and/or synthetic data, activity distributions and attenuation. A GAN may be trained to synthesize 3D MRI images, which can then be used as an additional step to generate activation and attenuation maps used for SPECT simulation. However, techniques discussed herein enable the direct translation from one image modality like CT or MRI, to another, e.g., SPECT. Moreover, techniques described herein may use real patient imaging data in combination with information of a simulation model, which improves model performances and robustness as well as allowing for smaller training data sets. In addition, by conditioning the result on the model output, the image generation may be guided with much more finesse and towards objectives that are beyond what is possible with existing approaches. Another approach is to train a CycleGAN to synthesize SPECT activity distribution or attenuation maps from MRI data. In one approach, a GAN may be used to translate raw (‘for-processing’) medical images to processed (‘for-presentation’) medical images, with particular attention to the breast. A similar approach consists of training a cGAN with architecture derived from pix2pix to translate from CT axial slices into perfusion CT/SPECT axial slices. In another technique, virtual lung SPECT/CT fusion images for functional avoidance radiotherapy planning may be generated using machine learning algorithms. In this case, CT and CT/SPECT image pairs for training and testing may be selected and pre-processed to be aligned. Slices that do not include lung parenchyma may be manually excluded. For techniques discussed further herein, the addition of the information from a lung simulation model to the input of the generative model allows improved robustness and performance. Moreover, two-dimensional (2D) machine learning models present problems when trying to generate 3D images. This may be because, when working on individual 2D slices, the model has no information concerning the adjacent slices. An additional advantage is the possibility of not having to exclude slices that do not include lung parenchyma, with the effect of further increasing model robustness.

GANs may be discussed for use with techniques herein. GANs may have certain advantages, such as high visual realism of detailed anatomical structures, strong image-to-image translation allowing better cross-modality translation or time-series synthesis, particularly cGANs. GANs may complete iterative models more quickly, allowing for more efficient clinical or real-time usage than diffusion networks. Diffusion models may also be used with techniques herein.

Diffusion models (like DDPM or cDDPM) may generate images through gradual denoising, avoiding training instability and mode collapses that may be seen in GANs. This may produce smoother, more consistent results. Diffusion models may approximate the entire data distribution and thus capture more subtle anatomical and physiological variation than GANs, which may be important to determine patient-specific or pathology-specific features.

Other generative models may be used with techniques discussed herein, such as variational autoencoders (VAE). VAEs may have the downside of producing more blurry or low-contrast images. Normalizing Flow Models may be used, although those techniques may not allow for a high degree of scalability, and may be computationally heavy for 3D volumes. Transformers may also be used, although very large data sets may be needed for training.

As discussed, a conditional diffusion denoising probabilistic model (cDDPM) may be used as a generative model, which is limited to the CT to MRI image-to-image translation task. The cDDPMs in this approach may be based on diffusion denoising probabilistic models (DDPMs). The image-to-image translation translates one imaging modality to another and might not enable the generation of a second image in which the organ or the patient may be in a different state or condition, i.e., the image-to-image translation might work only for the imaged state, as the first image may be the only input to the generative process. Moreover, this approach may require that the mapping between modalities can be learned from pairs of images acquired with different modalities. For this to work, the mapping between the two modalities cannot be influenced by further external factors that are not part of the first image. Consider, for example, the translation between a CT image and a SPECT image that results from the inhalation of radiolabeled aerosol. The SPECT image will be massively influenced by factors like the applied inhalation maneuver or device, as well as aerosol size distribution. Since information about these factors might not be included in the first CT image, standard image-to-image translation approaches will not work in this setting. Aside from this specific example, there may be many more where additional information is needed for high quality and reliable image-to-image translation. Thus, techniques presented herein may focus on the task of translating a medical image (for example, a 3D CT) to a SPECT image while considering additional inputs consisting not only of an image captured with another modality, but also information from a lung simulation model to improve performance and robustness of the trained generative models.

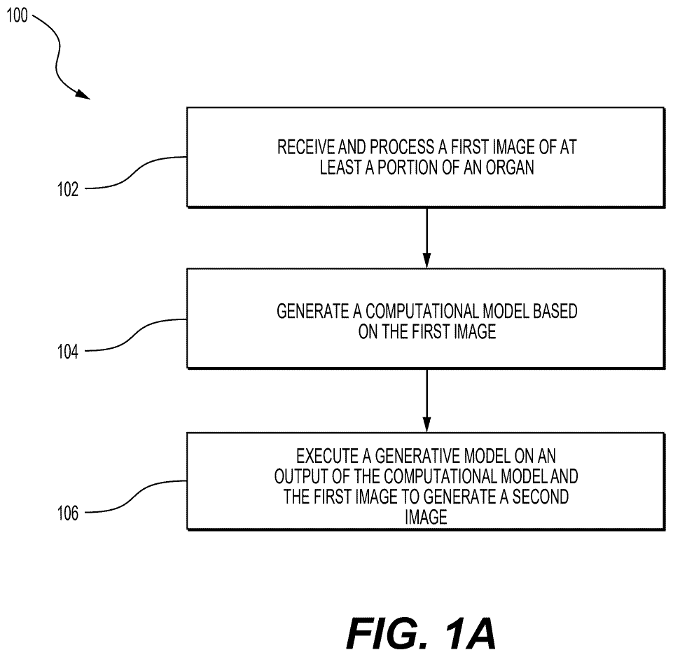

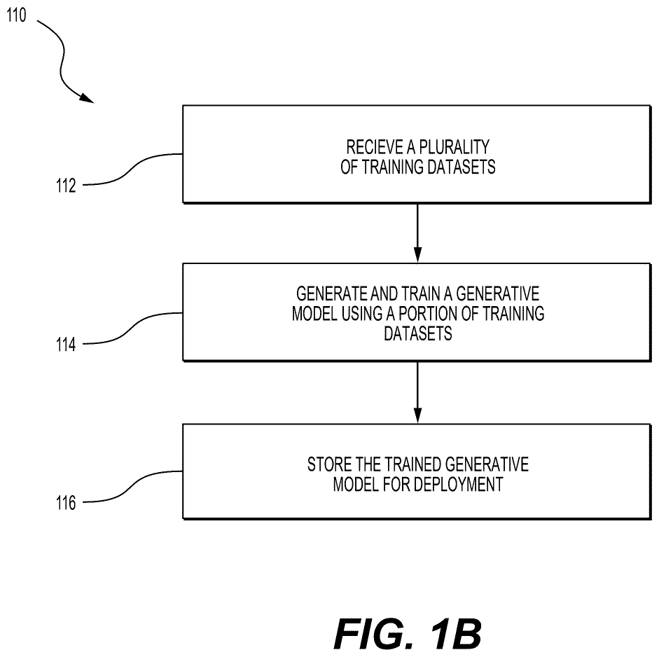

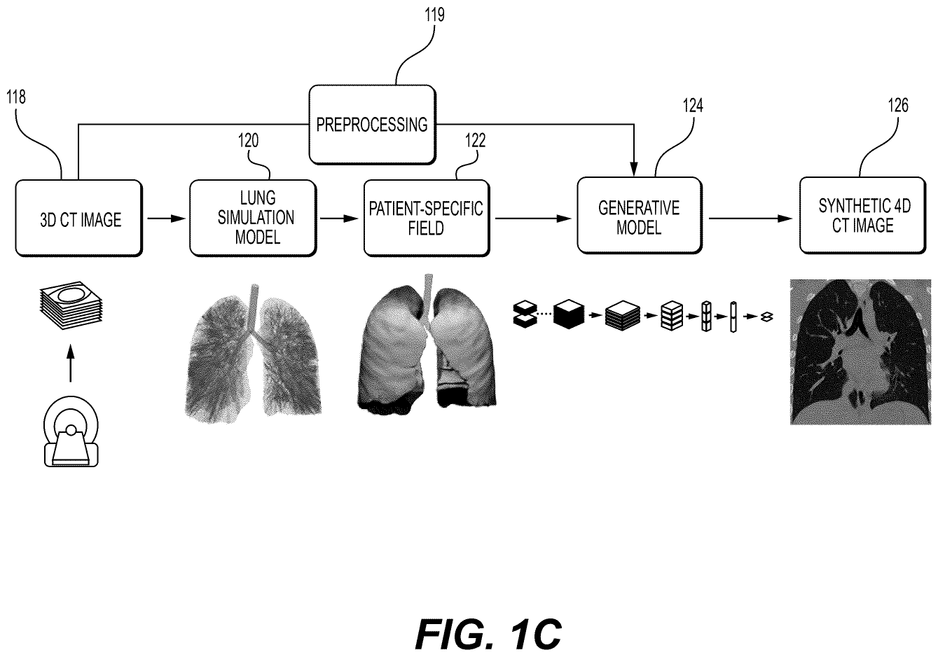

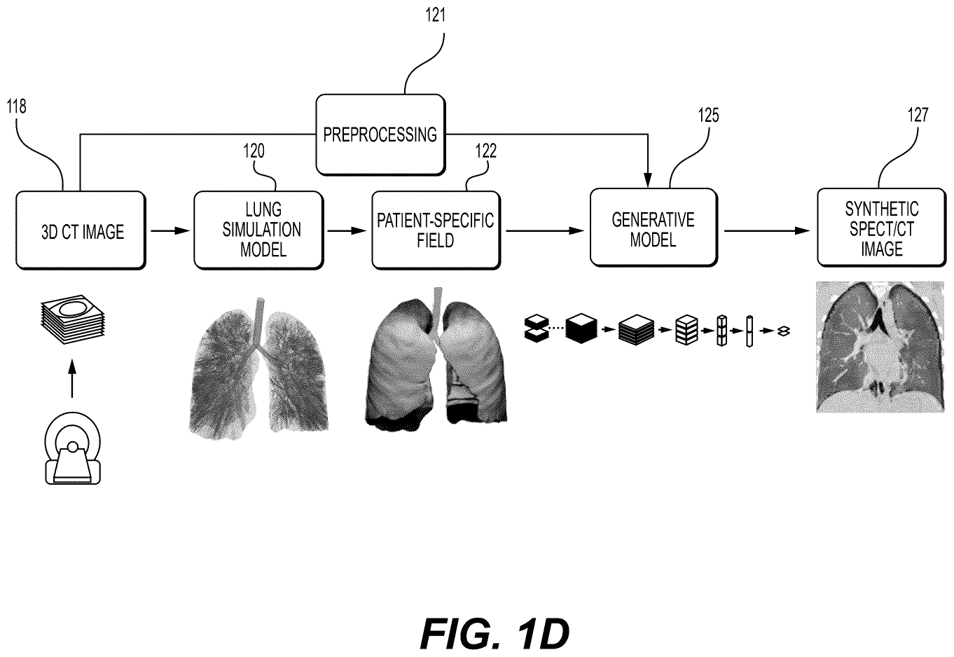

In these techniques, an entire respiratory cycle may be simulated (inhalation plus exhalation) with the lung simulation model. Information from the lung simulation model may be used, e.g., the strain or particle deposition field, at different respiratory states or the particle deposition field at end-expiration. A conditional generative model may then infer a synthetic 4D CT or SPECT image based on these inputs. To generate a synthetic 4D CT image, these techniques may combine the 3D CT scan with a time-resolved strain field computed by the lung simulation model and use a cGAN as the generative model (see FIG. 1C). To generate a synthetic SPECT image, the 3D CT scan may be combined with the strain or particle deposition field computed by the lung simulation model. A cGAN or a conditional diffusion denoising probabilistic model (cDDPM) may be used as generative model (see FIG. 1D). Alternative (e.g., conventional) techniques described above rely on purely data-driven generative models to synthesize images. The techniques described herein further condition the generative models on the output of a physics-based lung simulation model to improve the accuracy of the synthesized images, while allowing new use cases that enable not only a modality transform but also the generation of images under conditions that have not been imaged at all.

These techniques may use a single static 3D CT image. From this image, the patient-specific geometry of the respiratory system may be extracted. A physics-based lung simulation model comprising a flow simulation and/or a particle simulation may output a patient-specific scalar field with the same dimensions as the single static 3D CT image used as input. The scalar field may contain local information (e.g., on the alveolar cluster level) on one of the possible output quantities of the lung simulation model. These output quantities may be strain and/or particle deposition, but might also include stress, strain energy density, or power. Subsequently, this output of the lung simulation model and the original 3D CT image may be passed as input to a generative model trained to generate synthetic 4D CT or SPECT images. This may provide valuable information without exposing the patient to additional radiation, and it avoids the complex and time-consuming image acquisition process. Moreover, including physics-based information, i.e., the strain or particle deposition field, may improve the performance and the robustness of these techniques and the ability to generalize the model. The term robustness may mean the ability of a machine learning model to present valid outputs, even if the input presents perturbations or variations. This may be of particular relevance for machine learning applications with medical images, which can suffer from the limited amount of available medical data. Considering the variety that medical images can present, due to different anatomical characteristics, pathologies, and imaging acquisition methods, improving robustness may be important.

The workflow may consist of two major components: first, the physics-based lung simulation model, and second, the conditional generative model. Both components may operate on a single 3D CT image of the respiratory system as input. Alternatively, other imaging modalities, including magnetic resonance imaging (MRI), ultrasound, X-ray, or electrical impedance tomography (EIT), can be used. The respiratory system can be healthy or diseased. In particular, the single image may represent only one state (inhaled, exhaled, or in-between) of the respiratory system at a single point in time.

Additional inputs may include a breathing or ventilation curve, such as an individual inhalation and/or exhalation gas flow, a lung volume, or a lung pressure over time, for example over at least one full respiratory cycle. Under this premise, it should be highlighted that every possible respiration maneuver, either by spontaneous breathing or through a ventilator, may be able to be simulated by the lung simulation model. Additional input may also include properties of the inhaled aerosol and the inhaler, for example the mass or size distribution of the inhaled particles.

FIG. 1A depicts an example process 100 for generating synthetic images of an organ. FIG. 1B depicts an example process 110 for training a generative model for use in the example process of FIG. 1A. FIG. 1C shows a schematic illustration of the use of a first generative model 124 trained in accordance with FIG. 1B to generate a synthetic 4D CT image 126 using the process 100. FIG. 1D shows a schematic illustration of the use of a second generative model 125 trained in accordance with FIG. 1B to generate a SPECT/CT image 127 using the process 100.

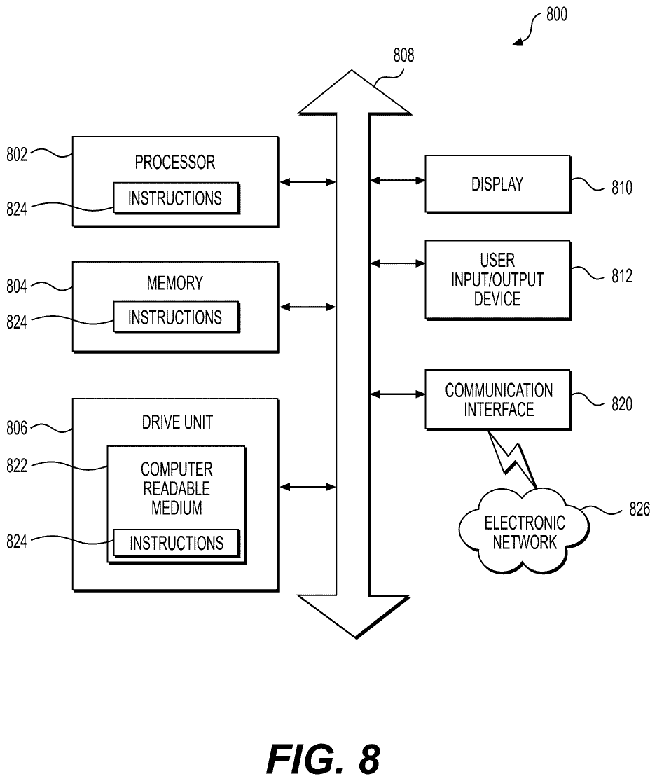

Referring to FIGS. 1A-1D concurrently, the example process 100 may be performed or implemented by one or more machines, such as a computing device or system described with reference to FIG. 8.

In step 102, the process 100 may include receiving and processing a first image of at least a portion of an organ. The first image may be obtained or captured by a medical imaging device using a first image modality. For example, the first image may be a 3D CT image captured by a CT imaging device. The 3D CT image may be a reconstructed image that is generated based on a plurality of 2D CT slices captured by the CT imaging device (e.g., as part of a 3D CT scan). The term 3D CT scan may be used synonymously with the term 3D CT image throughout this disclosure. In some examples, the first image may be a single 3D CT image. Alternatively, other imaging modalities, including magnetic resonance imaging (MRI), ultrasound, X-ray, or electrical impedance tomography (EIT), can be used to obtain the first image. The first image may be of a respiratory system of a patient and thus may be a patient-specific image. The respiratory system can be healthy or diseased. In particular, the first image may represent only one state (inhaled, exhaled, or in-between) of the respiratory system at a single point in time.

In step 104, the process 100 may include generating a computational model based on the first image. The computational model may be configured to simulate and/or predict one or more functional, mechanical, chemical, biological, and/or physiological quantities across a domain of at least the portion of the organ. In the examples described herein, the computation model may be a lung simulation model 120.

### I. Lung Simulation Model

The lung simulation model 120 may use the static 3D CT image 118 as input and compute a patient-specific strain or particle deposition field (e.g., patient-specific field) as an output 122. This model is included in PCT/EP2024/060180, which is incorporated by reference, to assess the efficacy of pulmonary drug delivery, with particular attention to regional deposition of an orally inhaled drug product. It may include the simulation of inhaled particle transport, absorption, and deposition in a human respiratory system following the approach disclosed in PCT/EP2021/059145, which is incorporated by reference. In this context, inhaled drugs are drugs that are only (intentionally) inhaled orally, but might not include drugs that are predominantly and exclusively administered nasally.

The lung simulation model 120 may consist of a flow simulation and, potentially, a particle simulation. The geometry used for these simulations may be based on segmentations of the lungs, lobes, initial parts of the airway tree, and its centerline, which are extracted from the static 3D CT image 118. The segmentation can be performed through computer vision and deep learning techniques. Due to resolution limitations, it may be generally impossible to extract the entire airway tree from the 3D CT image 118. Therefore, higher-generation airways below the image resolution may be added using a recursive space-filling tree growth algorithm, which results in a hybrid patient-specific/morphometric airway tree. This highly patient-specific geometry of the lungs and airway tree may allow modeling airflow within the airways and alveoli, as well as the elastic interaction with the ribcage and the diaphragm. The material properties may be calibrated using information from the 3D CT image 118, functional data from experimental datasets, and population averages.

In some aspects, the flow simulation may compute the airflow distribution throughout the respiratory system, e.g., as a result of a breathing/ventilation curve provided as a boundary condition. Computation of the transient airflow in the airway tree may be based on a reduced-dimensional formulation, e.g., by integrating the Navier-Stokes equations over the domain, exploiting information about the geometry of the airways, as well as the flow within them. Elasticity of airway walls may have a negligible influence on particle deposition results and may therefore be neglected. Elastic recoil of the chest wall and diaphragm as well as gravitational forces are accounted for using an external pressure boundary condition acting on the alveolar clusters. This pressure boundary condition depends on the current volume of the lung model and the weight of the lungs, as determined from the 3D CT image 118 using a density and volume analysis.

The output of the flow simulation may be the strain of each alveolar cluster. The strain may be calculated as the percentage change from a reference volume (i.e., volume of the alveolar cluster in a stress-free state) to the current volume at a given point in time. However, one alveolar cluster consists of multiple voxels of the 3D CT image 118, i.e., all voxels pertaining to a specific terminal airway may be assigned to the attached alveolar cluster. To obtain the strain for each CT voxel, the strain value calculated for the alveolar cluster may be applied to all assigned voxels. This means that all characteristics of an alveolar cluster equally apply to each voxel assigned to this alveolar cluster.

The results of the flow simulation may then be used for the particle simulation to compute the full trajectory of the inhaled particles as they are transported through the airway tree and potentially deposited at an airway wall, in the respiratory zone, or are exhaled. The particles may be modeled as point masses with a spherical shape. To simulate their transport, gravitational forces may be considered, as well as flow resistance based on the Reynolds number, and a buoyancy force due to the density differences between the particle and the fluid. The resulting system of ordinary differential equations may be solved using a forward Euler time-integration scheme. To compute the forces on the particles resulting from the fluid flow, the instationary reduced-dimensional flow field obtained from the patient-specific flow simulations may be leveraged to reconstruct the 3D fluid velocity field within each airway element. Particle transport across airway bifurcations may be computed using an interpolating surrogate model that is based on pre-computed local-scale 3D computational fluid and particle dynamics simulations of a representative airway bifurcation library. Briefly, 3D flow simulations may be conducted for a large library of airway bifurcations accounting for various flow regimes and geometries and subsequently simulate particle transport and deposition within these flow fields. The behavior of particles flowing across these airway bifurcations may be recorded, analyzed, and condensed into an interpolating surrogate model that may be used to compute particle transport across airway bifurcations in the lung simulation model 120. Particle deposition in the conducting airways may be assumed to occur on contact of the particle with the airway wall. If the particle enters an alveolar cluster, a deposition location within this alveolar cluster may be chosen randomly.

Returning to FIG. 1A, in step 106 the process 100 may include executing a generative model on an output of the computational model and the first image to generate a second image of at least the portion of the organ. When the computational model is the lung simulation model 120, the output 122 of the lung simulation model 120 may be the above-described patient-specific strain or particle deposition field that is provided as input to a generative model along with the 3D CT image 118. The second image may be a synthetic image that is synthesized or translated. In some examples, both types of second images may be generated (e.g., at least two images are generated).

A type of generative model executed may be dependent on the type of the second image to be generated. As one example, the second image may be a 4D CT image 126. In some examples, the 4D CT image 126 may be generated under different real or hypothetical/simulated conditions, including different time, different mechanical, or physiological conditions of the first imaging modality (e.g., CT imaging). The 4D CT image 126 may be generated using a first generative model 124, such as a cGAN (see FIG. 1C). In other examples, the second image may be a SPECT/CT image 127 having a different, second modality (e.g., nuclear imaging). The SPECT/CT image 127 may be generated using a second generative model 125 (see FIG. 1D). In some embodiments, the second generative model 125 may be a same type of model as the first generative model 124 (e.g., a cGAN). In other embodiments, the second generative model 125 may be a different type of model, such as a cDDPM. Each type of second image generation using the various generative models is addressed in turn below.

Process 100 described above is provided merely as an example, and may include additional, fewer, different, or differently arranged steps than depicted in FIG. 1A.

II. Synthetic 4D CT Image Generation with a Conditional Generative Adversarial Network

The term “machine learning model” may refer to a machine or deep learning model configured to receive one or more inputs and yield one or more outputs based on a model architecture, training data, inference procedures, or other information acquired while training the model. The machine learning model may be useful for predicting or inferring outputs based on user input. The machine learning model may be coupled to, or integrated with, one or more medical imaging devices, e.g., for executing the machine learning model with/on one or more medical images captured or otherwise acquired by the medical imaging devices.

The term “generative model” may refer to a machine learning model that learns the underlying distribution or relationships within training data and can generate new, synthetic examples that resemble the original data.

The term “architecture” may refer to the sequence of layers, connections, and components that describe the data flow within the model.

The term “objective function” may refer to a function used in model training that quantifies the accuracy of the model's predictions against the expected output. The objective function may be minimized or maximized while training the machine learning model, e.g., depending on specific function selected.

The term “convolutional layer” may refer to a layer in the model architecture in which a convolution kernel is convolved with its input over a single dimension to produce an output. In this context, the kernel size specifies the dimension of the convolutional window, and stride refers to the step size with which the convolution kernel moves across the input data.

The term “padding” may refer to adding non-relevant information around the borders of the input before performing a convolution, e.g., such that the output has the same dimensions as the input.

The term “transpose convolutional layer” may refer to a layer in which the transformation performed is in the opposite direction of the convolutional layer. For example, from an image that has the shape of the output of a given convolution to another image that has the shape of its input while maintaining a connectivity pattern compatible with that convolution, similar for kernel size, stride, and padding.

The term “channels c” may refer to the last dimension of an image. For example, a two-dimensional (2D) image can be expressed as (H, W, c); where H equals height and where W equals width (of the 2D image). For the case of 3D CT images given as part of the machine learning model input, these will present C channels.

As shown in FIG. 1C, the input of a machine learning model comprising the first generative model 124 may be the 3D CT image 118, x∈ [symbol] ³, combined with the output 122, aᵢ∈ [symbol] ³, of the lung simulation model 120, which, in this case, is the strain or deposition field (e.g., the patient-specific field) evaluated at different respiratory states, i.e., N specific points in time tᵢ with i=1, . . . , N for which an inhalation experiment has been previously carried out. Together, the 3D CT image 118 and the output 122 of the lung simulation model 120 may form the input [symbol] (x, aᵢ) of the machine learning model. In some examples, the 3D CT image 118 may undergo preprocessing 119 prior to forming the input [symbol] (x, aᵢ). As presented in FIG. 1C, the input [symbol] (x, aᵢ) may be used to condition the first generative model 124 to generate the synthetic 4D CT image 126 as output [symbol] *. The output [symbol] * may be a sequence of N generated 3D images

$${Y}_{i}^{*}$$

$${y}^{*} = \{ {Y}_{1}^{*} ( 𝒥 ( x , {a}_{1} ) ) , {Y}_{2}^{*} ( 𝒥 ( x , {a}_{2} ) ) , … , {Y}_{N}^{*} ( 𝒥 ( x , {a}_{N} ) ) \} = {\{ {Y}_{i}^{*} ( 𝒥 ( x , {a}_{i} ) ) \}}_{i = 1}^{N} .$$

All the generated images

$${Y}_{i}^{*}$$

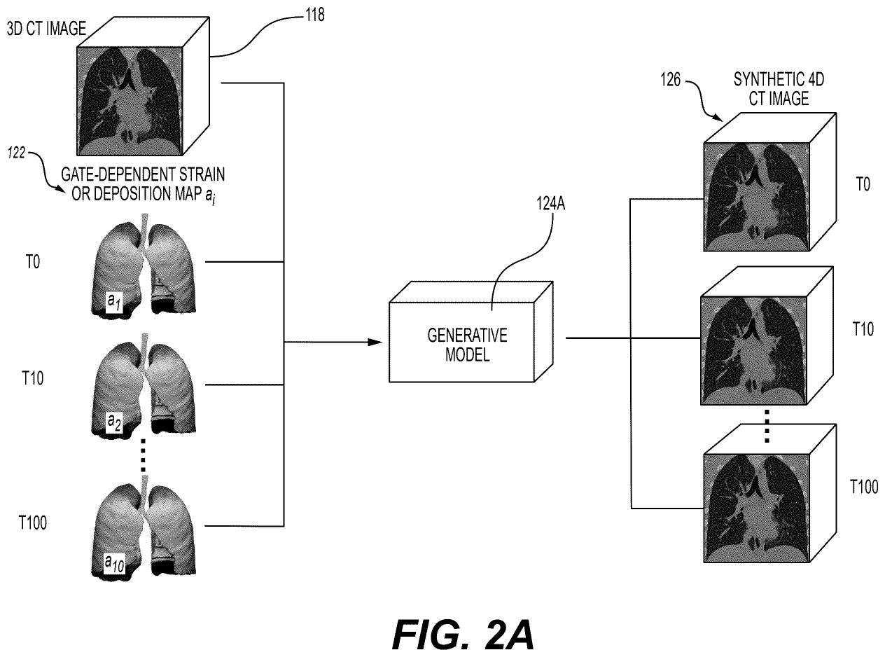

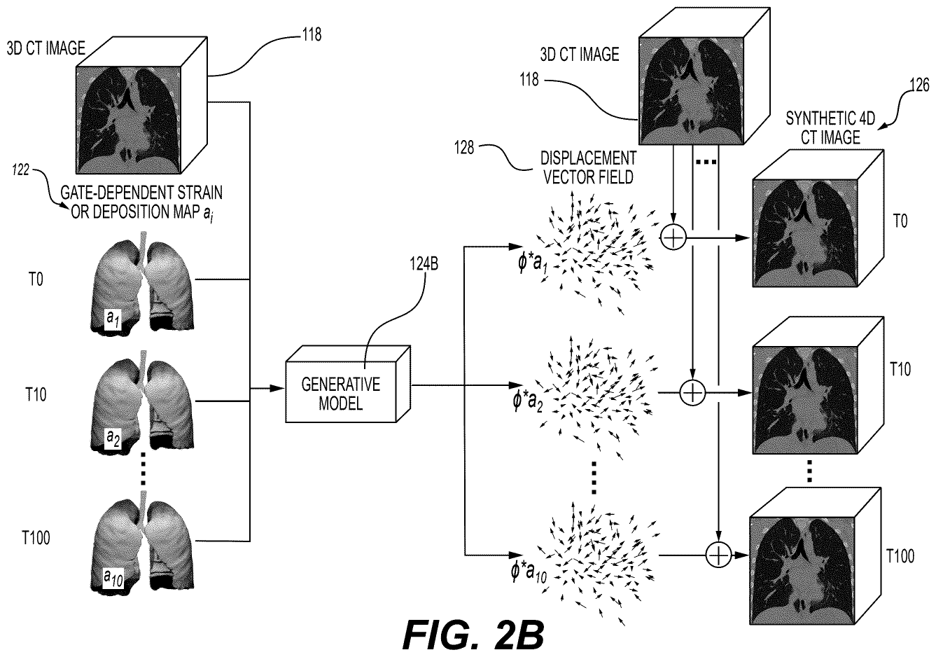

comprising the synthetic 4D CT image 126 are associated with the same point in time t₁ of the strain or deposition field at that are used as part of the input (e.g., see FIGS. 2A and 2B for an illustration of the associated time points: T0, T10, . . . , T100). The 3D CT image 118, x, may also refer to one of said points in time t; and can be associated with one of the possible aᵢ. Furthermore, this can be chosen in one arbitrary point of the simulated breathing cycle (e.g., beginning of inspiration or end-expiratory state) and therefore the generated images can contain time information precedent, coincident, or following the 3D CT image 118, x.

The lung simulation model 120 may be a dynamic model, and hence its output 122, at, can be computed for arbitrary points in time, used to condition the first generative model 124, and generate the corresponding image

$${Y}_{i}^{*} .$$

The employed first generative model 124 may be a cGAN, which is a deep-learning architecture that learns a mapping from the input [symbol] (x, aᵢ) and a random noise vector z to an output

$${Y}_{i}^{*} ,$$

so that

$$G : \{ 𝒥 ( x , {a}_{i} ) , z \} \to {Y}_{i}^{*} .$$

The cGAN may comprise two neural networks contesting with each other: a generator G and an adversarial-trained discriminator D. The generator may be trained to produce outputs

$${Y}_{i}^{*}$$

that cannot be distinguished from actual 3D CT images. The discriminator may be adversarially trained to detect the generator's synthetic images. Without the random noise vector z, the cGAN can still learn a mapping from the input [symbol] (x, aᵢ) to the output

$${Y}_{i}^{*}$$

but would produce deterministic output instead of stochastic output. This may be why Gaussian noise may be added as an input to the generator, but this may result in the generator learning to ignore the noise. Therefore, noise might be only included in the form of dropout, applied on several layers of the generator both for the training and testing. Accordingly, the random noise vector z will not be explicitly included in the notation.

The cGAN learns the mapping conditioned on the input [symbol] (x, aᵢ) to infer the output

$${Y}_{i}^{*} .$$

This may be achieved by using the cGAN for inference multiple times, each time based on the strain or deposition field (e.g., the output 122) at the corresponding point in time (see FIGS. 2A and 2B for an illustration of the different time points: T0, T10, . . . , T100). All these images will be collected to form one final 4D CT image 126. In other words, the 3D CT image 118 never changes, but the strain field (e.g., the output 122) varies to provide the model time-dependent information. The sequence of inferences forms the final time series [symbol] * of images similar to a real 4D CT image [symbol] .

Variants of the first generative model 124 may include a first variant 124A (Variant A) and a second variant 124B (Variant B), as illustrated in FIGS. 2A and 2B, respectively. In Variant A, a cGAN may directly generate multiple images, which then form the sequence of images of the synthetic 4D CT image 126. In Variant B, a cGAN may produce a displacement vector field (DVF) 128,

$${ϕ}_{a , i}^{*} ,$$

which is then employed to warp the input 3D CT image 118 to obtain images comprising the synthetic 4D CT image 126.

As described in more detail with reference to the training process 110 of FIG. 1B, the training and the test dataset for the cGAN may include real 4D CT images and corresponding lung simulation model outputs. Each real 4D CT image [symbol] consists of multiple 3D CT images Yᵢ acquired at a specific point in time. One point in time may be chosen as a reference to set up the lung simulation model 120. Together with the respiratory or ventilation curve, the lung simulation model output, i.e., the strain field, is computed for every 3D CT image contained in the series of images comprising the real 4D CT image. For Variant B, the training and test dataset additionally contains the displacement vector fields φᵢ for every Yᵢ. An image registration process may be used to derive the displacement vector fields φᵢ between a reference image and every other image of the real 4D CT image. In the following, the displacement vector fields φᵢ derived from image registration are used as ground truth.

Although the training and the test datasets are described as including real 4D CT images, in some examples, the 4D CT images used for training and testing may be synthetically generated 4D CT images (e.g., using the process 100) that have been confirmed or verified as accurate, for example.

### Variant A

The first variant 124A of the first generative model 124 (Variant A of the cGAN) is illustrated in FIG. 2A, and as mentioned above, the model directly infers the sequence of images forming the synthetic 4D CT image 126. The cGAN for Variant A may have an architecture derived from vox2vox and may be trained with an objective function that accounts for the training of both the generator and the discriminator.

$$ℒ = 𝔼 {‖ D ( 𝒥 ( x , {a}_{i} ) , {Y}_{i} ) - 1 ‖}_{2} + 𝔼 {‖ D ( 𝒥 ( x , {a}_{i} ) , {Y}_{i}^{*} ) ‖}_{2} + {‖ D ( 𝒥 ( x , {a}_{i} ) , {Y}_{i}^{*} ) - 1 ‖}_{2}$$

where ∥⋅∥₂ may be the L₂ norm. The expected values [symbol] are calculated across all dimensions. The output of the generator may be

$${Y}_{i}^{*} = G ( 𝒥 ( x , {a}_{i} ) )$$

based on the input [symbol] (x, aᵢ). The output of the discriminator may be D( [symbol] (x, aᵢ), Yᵢ) based on the input [symbol] (x, aᵢ) and real training image Yᵢ or

$$D ( 𝒥 ( x , {a}_{i} ) , {Y}_{i}^{*} )$$

based on the output of the

$${Y}_{i}^{*} .$$

To further enhance the physics-conditioned nature of the model, a component based on the L₂ distance between the real training image [symbol] and the generated image [symbol] * may also be possible.

### Variant B

The second variant 124B of the first generative model 124 (Variant B of the cGAN) is illustrated in FIG. 2B. In Variant B, the model may be also repeatedly used to infer the entire respiratory cycle based on the input [symbol] (x, aᵢ). The 3D CT image 118, x, may be constant, and the lung simulation model output 122, aᵢ, (e.g., strain field) may be variable to provide the model time-dependent information. The architecture may again be derived from vox2vox, but here, the approach differs from that of Variant A. The fundamental difference may be that the model output is not directly a synthetic 3D CT image as part of the final series [symbol] * of CT images. Rather, the model may infer the displacement vector fields (DVFs) 128,

$${ϕ}_{a , i}^{*} ,$$

which resemble the ground-truth DVFs φᵢ. Since the model output may be the inferred DVFs 128 (see FIG. 2B), the generated image may have 3 channels to account for all spatial dimensions of the DVFs. The inferred DVFs 128,

$${ϕ}_{a , i}^{*} ,$$

may then be used to warp the 3D CT image 118, x, resulting in the output image

$${Y}_{i}^{*} = x ∘ {ϕ}_{a , i}^{*} .$$

This gives the final sequence of images that comprise the synthetic 4D CT image 126. The warping process may use a spatial transformer, such as Elastix, ANTs, or pTVreg. Another difference may be that because the ground-truth DVFs φᵢ and the inferred DVFs 128,

$${ϕ}_{a , i}^{*} ,$$

are also available, they can be concatenated to the input of the discriminator. The rationale behind this is that the information contained in the magnitude relates to the motion amplitude of the patient's respiratory pattern. For example, D( [symbol] (x, aᵢ), Yᵢ, ∥φᵢ∥) is the output of the discriminator using the input [symbol] (x, aᵢ), the real training image Yᵢ, and the magnitude of the ground-truth DVF φᵢ. The objective function for Variant B may be a combination of supervised and unsupervised components given by

$$ℒ = {λ}_{1} ( {\sum }_{x , y , z} {‖ {ϕ}_{a , i}^{*} - {ϕ}_{i} ‖}_{1} ) + {λ}_{2} \{ 𝔼 [ log D ( 𝒥 ( x , {a}_{i} ) , {Y}_{i} , ‖ {ϕ}_{i} ‖ ) ] + 𝔼 [ log ( 1 - D ( 𝒥 ( x , {a}_{i} ) , {Y}_{i}^{*} , ‖ {ϕ}_{a , i}^{*} ‖ ) ) ] \}$$

where the parameters λ₁ and λ₂ balance the two components. The expected values [symbol] are calculated across all dimensions. The first term, weighted with λ₁, may be a L₁ reconstruction norm summed over all spatial components x, y, z. To encourage the generation of realistic DVFs without explicitly modeling field smoothness, the second term, weighted with λ₂, is an adversarial objective that accounts for the warped images

$${Y}_{i}^{*}$$

and the magnitude of the DVFs.

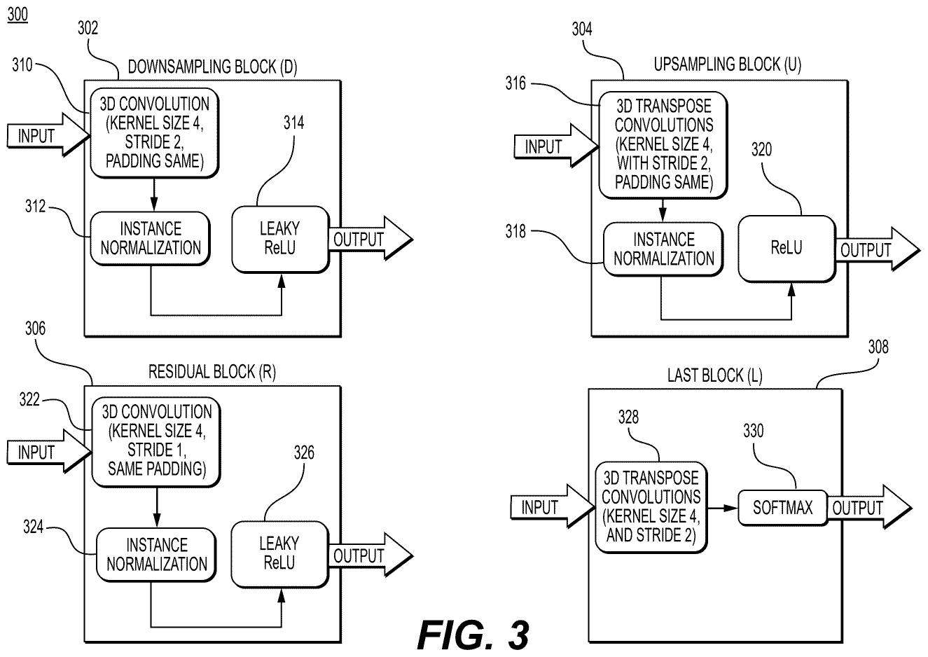

An architecture of the cGAN for either of Variant A and Variant B, described above with reference to FIGS. 2A and 2B, respectively, may be derived from a vox2vox architecture. The architecture comprises several types of functional blocks 300, illustrated in FIG. 3, that form the functional units of the model, are repeated through the architecture, and have specific purposes within the architecture. Turning now to FIG. 3, the functional blocks 300 may include a downsampling block 302 (D), an upsampling block 304 (U), a residual block 306 (R), and a last block 308 (L).

In particular, the downsampling block 302 (D) may consist of a 3D convolutional layer 310, instance normalization 312, and/or leaky rectified linear unit (ReLU) 314. The 3D convolutional layer 310 may have a kernel size of 4, a stride of 2, and same padding. At the end of each block, the number of output channels may be doubled with respect to the input. Only the first downsampling block has an output with 64 channels.

The upsampling block 304 (U) may consist of a 3D transposed convolutional layer 316, instance normalization 318, and a ReLU 320. The 3D transposed convolutional layer 316 may have a kernel size of 4 and a stride of 2. At the end of each upsampling block 304, the number of the output channels may be halved compared to its inputs.

The residual block 306 (R) consists of a 3D convolutional layer 322, instance normalization 324, and leaky ReLU 326. The 3D convolutional layer 322 may have a size 4 kernel, a stride of 1, and same padding.

The last block 308 (L) may consist of a 3D transposed convolutional layer 328 and a softmax activation function 330. The output of the last block 308 may be the output of the generator and has the same number of channels, C, as the 3D CT image 118 that forms part of the input.

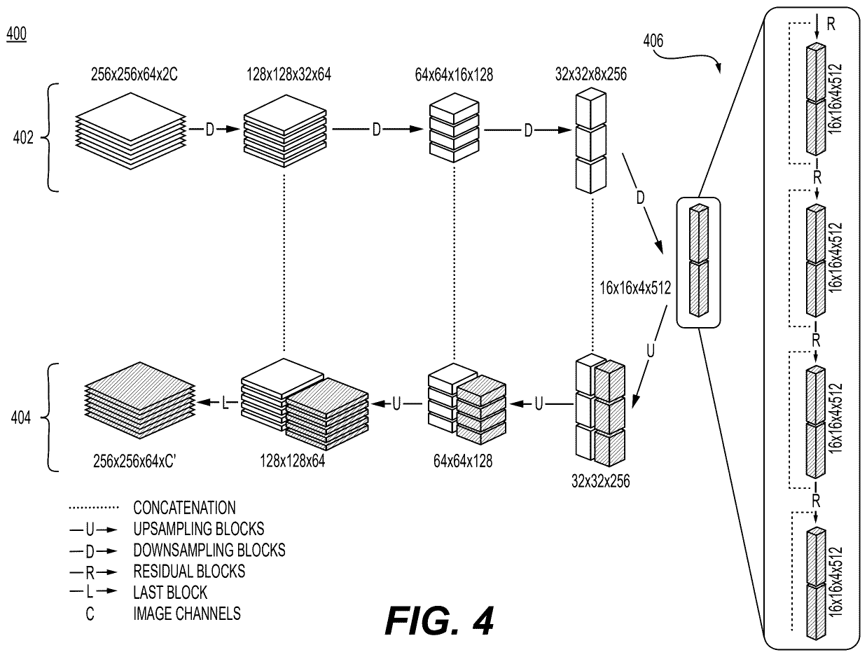

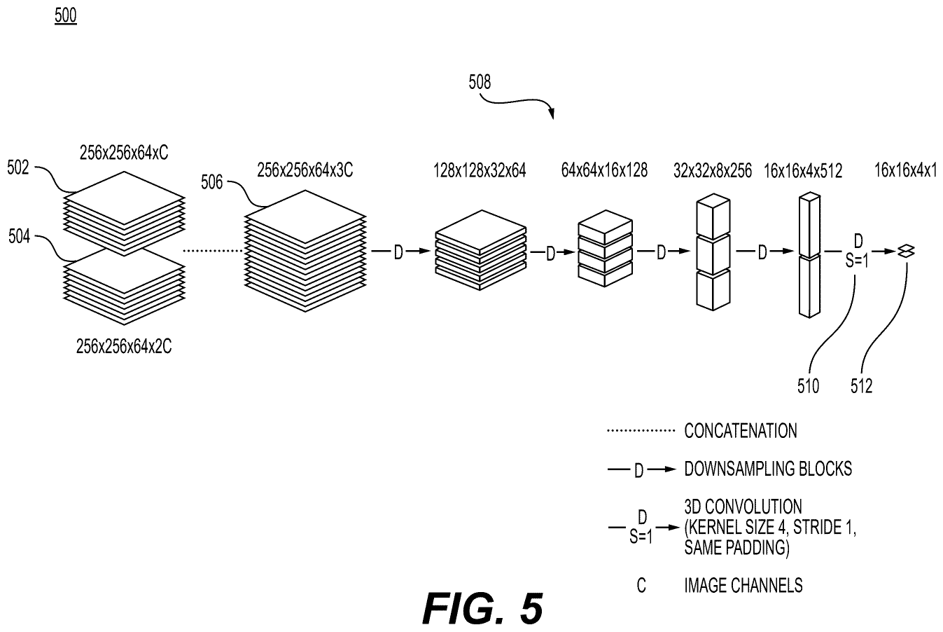

FIGS. 4 and 5 illustrate architecture of the cGAN according to aspects of the present disclosure.

As shown in FIG. 4, the generator of the cGAN of either Variant A or Variant B may have a U-Net architecture 400, e.g., such that the architecture takes the form of a “U” as shown, including an encoding (or “contracting”) path 402, a residual block application 406, and a decoding (or “expanding”) path 404. For example, on the encoding path 402, four downsampling blocks may be applied iteratively such that the number of channels may be doubled at each iteration. The downsampling blocks may be the same or similar to the downsampling block 302 described with reference to FIG. 3. Between the encoding path 402 and decoding path 404, four residual blocks may be applied repeatedly as part of the residual block application 406. The residual blocks may be the same or similar to the residual block 306 described with reference to FIG. 3. Here, the output from each residual block may be concatenated with its input before the successive residual block may be applied. On the decoding path 404, the output of each upsampling block 304 may be concatenated with the output of the corresponding downsampling block in the encoding path 402, forming the input of the next upsampling block. The upsampling blocks may be the same or similar to the upsampling block 304 described with reference to FIG. 3. Finally, a last layer or last block (e.g. the same or similar to the last block 308 described with reference to FIG. 3) may generate the desired output image.

The discriminator of the cGAN of either Variant A or Variant B may have a PatchGAN architecture 500, as presented in FIG. 5. PatchGAN may be used to infer whether overlapping image patches of dimensions R×R (typically R=70) are real, focusing on the local structure of the image. Therefore, the discriminator may be run convolutionally across the image, and the responses are averaged across all output dimensions. First, the input of the vox2vox model 502 and the generator output 504 are concatenated (e.g., to generate a concatenated input 506), resulting in a total number of 3C channels. Then, four downsampling blocks 508 are applied, identical to those used in the generator. The downsampling blocks 508 may be similar to the downsampling block 302 described with reference to FIG. 3. Finally, a convolutional layer 510 with kernel size 4, stride 1, and same padding may be applied to obtain a final output 512 with 1 channel, representing the quality of the generated patch. The output image may have pixel values between 0 and 1, with each pixel representing the probability that each 70×70 patch may be taken from a real image. The encoding and decoding blocks of the first and last layers of the generator or the discriminator may have some exceptions and may consist only of convolutional layers.

III. Synthetic SPECT Image Generation with a Conditional Generative Adversarial Network

As shown in FIG. 1D, to generate a synthetic SPECT/CT image 127, the 3D CT image 118 may be used together with the output 122 by the lung simulation model 120 (e.g., the strain or particle deposition field) to condition the second generative model 125. The synthetic SPECT/CT image 127 may be obtained either as the direct output of the second generative model 125 or as the result of a post-processing step of the generative model's output. In the latter case, the model output may be a SPECT image, which is fused with the input 3D CT image 118 to obtain the synthetic SPECT/CT image 127. In some examples, the second generative model 125 may be a cGAN, based on a vox2vox architecture and inputs similar to the inputs received in the first variant 124A and the second variant 124B of the first generative model 124 (e.g., Variant A and B of the cGAN), described above.

The input [symbol] (x, a) of the cGAN may consist of the 3D CT image 118, x, and the output 122, a, of the lung simulation model 120, either a strain field or a particle deposition field. In some examples, the 3D CT image 118, x, may undergo preprocessing 121 prior to forming the input [symbol] (x, a). Again, an entire respiratory cycle may be simulated with the lung simulation model 120 and either use the strain field at full inspiration or the particle deposition field at end-expiration. The output of the cGAN may be a 3D SPECT image. The architecture of the cGAN may be the same as for Variant A and B of the cGAN, illustrated in FIGS. 3, 4, and 5. Although the architecture may be the same, the cGAN may be different from the cGAN described above with reference to FIGS. 3, 4, and 5. For example, for the generation of the SPECT/CT image 127 with the second generative model 125, the cGAN may be trained with an objective function that accounts for the training of both the generator and the discriminator

$$ℒ = 𝔼 {‖ D ( 𝒥 ( x , a ) , Y ) - 1 ‖}_{2} + 𝔼 {‖ D ( 𝒥 ( x , a ) , {Y}^{*} ) ‖}_{2} + {‖ D ( 𝒥 ( x , a ) , {Y}^{*} ) - 1 ‖}_{2} ,$$

where ∥⋅∥₂ is the L₂ norm. The expected values [symbol] are calculated across all dimensions. The output of the generator may be Y*=G( [symbol] (x, a)) based on the input [symbol] (x, a). The output of the discriminator may be D( [symbol] (x, a),Y) based on the input [symbol] (x, a) and real training image Y or D( [symbol] (x, a), Y*) based on the output of the generator Y*. To further enhance the physics-conditioned nature of the model, a component based on the L₂ distance between the real training image [symbol] and the generated image [symbol] * is also possible.
IV. Synthetic SPECT Generation with a Conditional Diffusion Denoising Probabilistic Model

In other examples, the second generative model 125 used to generate the synthetic SPECT/CT image 127 may be a conditional diffusion denoising probabilistic model (cDDPM), another type of generative model. Diffusion denoising probabilistic models (DDPM) may be formulated as parameterized Markov chains and trained using variational inference to produce samples matching the data after finite time. They may consist of two stages: a forward diffusion process and a reverse process. The forward diffusion process gradually adds Gaussian noise to the image using multiple steps of a parametrized Markovian process. The reverse process may iteratively denoise the target image. For cDDPMs, the reverse stage may be conditioned on a source image. Here, the cDDPM may be doubly conditioned on the input 3D CT image 118 and corresponding output 122 of the lung simulation model 120 which here is the particle deposition field.

The dataset comprises N input-output pairs

$$𝒟 = {\{ 𝒥 ( {x}^{j} , {a}^{j} ) , {Y}^{j} \}}_{j = 1}^{N} ,$$

where x^{j} is the input 3D CT image 118, a^{j} the corresponding output 122 of the lung simulation model 120, and Y^{j} is the output, formed by the target SPECT image and the same output 122, a^{j}, of the lung simulation model 120 concatenated together. Again, an entire inhalation experiment may be simulated with the lung simulation model 120 and either use the strain field at full inspiration or the particle deposition field at end-expiration as the output 122, a^{j}, of the lung simulation model 120. Here, j=1, . . . , N is the index of the input-output pair.

The forward diffusion process may be a Markovian process that gradually adds Gaussian noise to the image Y₀ over T iterations according to a variance schedule

$${β}_{1} , … , {β}_{T}$$

$$q ( {Y}_{1 : T} \vert {Y}_{0} ) = {\prod }_{t = 1}^{T} q ( {Y}_{t} \vert {Y}_{t - 1} ) ,$$

$$q ( {Y}_{t} \vert {Y}_{t - 1} ) = 𝒩 ( {Y}_{t} ; \sqrt{1 - {β}_{t}} {Y}_{t - 1} , {β}_{t} I ) ,$$

resulting in a sequence Y₁, Y₂, . . . , Y_{T} of gradually corrupted images. [symbol] (Y; μ, σ) denotes a Gaussian distribution with mean μ and covariance σ, and βₜϵ(0,1) is a hyperparameter controlling the variance of incremental Gaussian noise. The final image Y_{T} is pure Gaussian noise, hence p(Y_{T})=N [symbol] (Y_{T}; 0, I).

In the reverse process, a machine learning model f_{θ} may be trained to approximate each reverse diffusion step based on estimating the noise vector ϵₜ given any noisy image. The reverse Markovian process may be given by

$${p}_{θ} ( {Y}_{0 : T} \vert 𝒥 ( x , a ) ) = p ( {Y}_{T} ) {\prod }_{t = 1}^{T} {p}_{θ} ( {Y}_{t - 1} \vert {Y}_{t} , 𝒥 ( x , a ) ) ,$$

$${p}_{θ} ( {Y}_{t - 1} \vert {Y}_{t} , 𝒥 ( x , a ) ) = 𝒩 ( {Y}_{t - 1} ; {\tilde{μ}}_{θ} ( {Y}_{t} , 𝒥 ( x , a ) ) , {\tilde{β}}_{t} I ) with$$

$${\tilde{μ}}_{θ} ( {Y}_{t} , 𝒥 ( x , a ) , t ) = \frac{1}{\sqrt{{α}_{t}}} ( {Y}_{t} - \frac{1 - {α}_{t}}{\sqrt{1 - {γ}_{t}}} {f}_{θ} ( 𝒥 ( x , a ) , {Y}_{t} , {γ}_{t} ) ) and {\tilde{β}}_{t} = \frac{1 - {γ}_{t - 1}}{1 - {γ}_{t}} {β}_{t} ,$$

$$where {α}_{t} = 1 - {β}_{t} , {γ}_{t} = {\prod }_{i = 1}^{t} {α}_{i} ,$$

and θ is the vector of parameters optimized during training.

The objective function for training the model may be given by

$$ℒ = {𝔼}_{t \sim [ 1 , T ] , {Y}_{0} , {ϵ}_{t}} [ {‖ {ϵ}_{t} - {f}_{θ} ( 𝒥 ( x , a ) , \sqrt{{γ}_{t}} {Y}_{0} + \sqrt{1 - {γ}_{t}} {ϵ}_{t} , {γ}_{t} ) ‖}^{2} ] + C ,$$

where C is a constant independent of the vector of parameters θ.

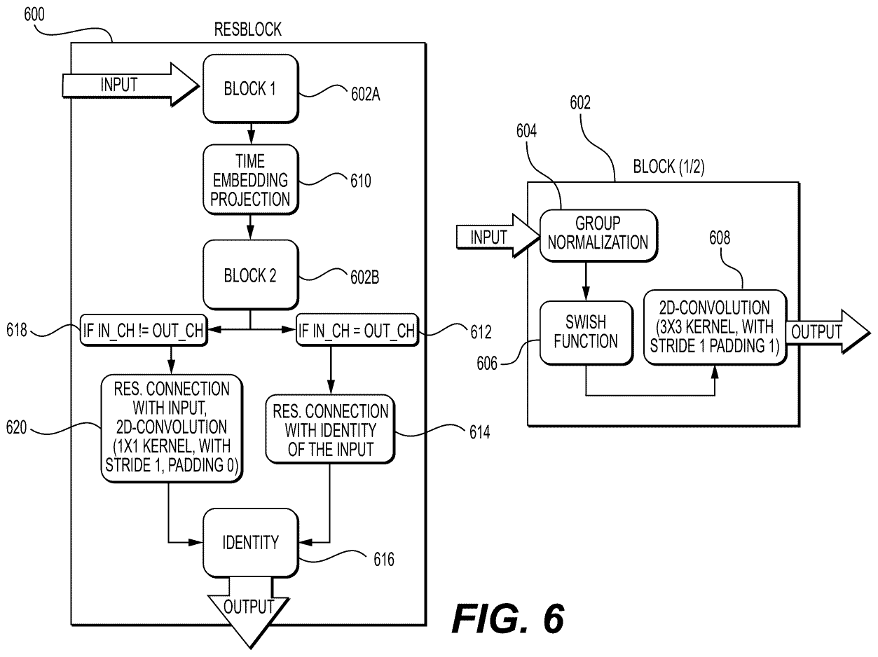

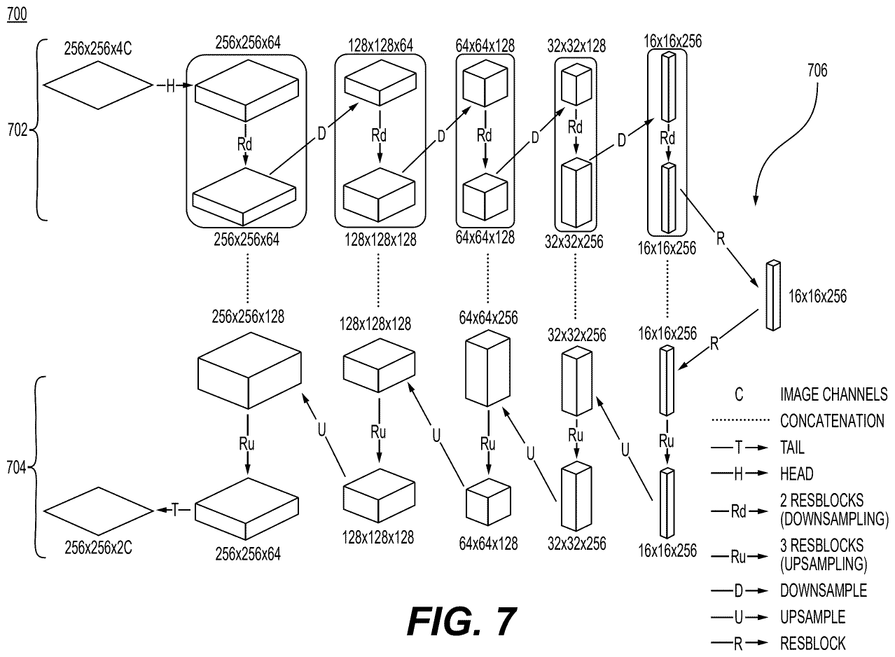

The architecture of the model used in the reverse process may be derived from a U-Net architecture 700, as shown in FIG. 7, and may be based on a composition of multiple functional blocks. The residual block (ResBlock) 600 depicted in FIG. 6 is one example core functional block of the U-Net architecture 700.

Turning to FIG. 6, the ResBlock 600 may include a first block 602A (Block 1) and a second block 602B (Block 2), collectively blocks 602. Each of the blocks 602 (e.g., Block 1/2 represented in detail on the right-hand side of FIG. 6) may be comprised of a group normalization 604, a Swish function 606, and 2D convolutional layers 608, with kernel size 3, stride 1, and padding 1. The blocks do not change the height and width of the image, but dropout could additionally be applied to the second block 602B before the last convolution.

The ResBlock 600 may also include a time embedding projection unit 610, which may be summed to the input after the first block 602A. The time embedding projection unit 610 may include a Swish function and a linear layer using a time embedding vector used to condition the model with the time t and takes the following form: (sin(2πWt), cos(2πWt)), where W is a random weight with a normal distribution with average 0 and standard deviation 1 that is sampled during the initializations and is trainable.

One of two residual connections (e.g., a first residual connection 614 or a second residual connection 620) may follow the second block 602B dependent on a determination of a number of the input channels dimension relative to the output. For example, if the number of input channels dimension is the same as the output (e.g., a first determination 612), the first residual connection 614 may follow and the input may pass through an identity layer before being added to the output. Otherwise, based on a second determination 618 that the number of input channels dimension is different from the output, the second residual connection 620 may follow and the input passes through a convolutional layer (with kernel size 1, stride 1 and padding 0) before being added to the output.

One final identity layer 616 may be applied to generate the final output of the ResBlock 600. Alternative implementations with attention blocks substituting the identity layers are also possible.

The U-Net architecture 700 is shown in FIG. 7. After concatenating the input [symbol] (x^{j}, a^{j}) with the output Y^{j}, the following steps may be sequentially applied. A head (H) starts the encoding path 702 of the U-Net architecture 700. The head may consist of a 2D convolutional layer with kernel size 3, stride 1, and padding 1. At the end of the head, the output's number of channels may be 64. The encoding path 702 further comprises the sequential application of groups of two residual blocks (Rd) and a sub-sequential downsampling block (D) for five and four times, respectively. Each residual block and the downsampling block may be similar to the residual block 306 and the downsampling block 302, respectively, as described above with reference to FIG. 3. In particular, the first residual block of each group changes the number of channels according to the pre-defined channel multipliers [1,2,2,4,4]. The channel multipliers express the multiplicative factor of the layer outputs channels relative to the one at the end of the head, which is 64. The other dimensions (height and width) are preserved. The downsampling blocks may preserve the number of channels, but the other output dimensions are halved. It may comprise a 2D convolutional layer with kernel size 3, padding 1, and stride 2. After the encoding path 702 and before the decoding path 704, two residual blocks (R) may be applied sequentially (e.g., a residual block application 706). As already mentioned, these residual blocks may preserve the dimensions of the image and constitute the bottleneck of the U-Net architecture 700. The decoding path 704 may be formed by the sequential application of groups of three residual blocks (Ru) and an upsampling block (U) for five and four times, respectively. The upsampling block may be similar to the upsampling block 304 as described above with reference to FIG. 3. Again, the first residual block of the group may change the number of channels according to the pre-defined channel multipliers while preserving the other dimensions. Moreover, for each residual block of the decoding path 704, the output from the previous block (whether upsampling or residual) may be concatenated with the corresponding, symmetrical output from the encoding path 702 (which can be either a residual or a downsampling block). Consequently, the concatenation may involve all the residual blocks (Ru and Rd) and the downsampling blocks, as marked by the rounded rectangles. The upsampling blocks preserve number of channels but doubles the height and width. It may be formed by an interpolated upsampling with scale 2 and a 2D convolutional layer with kernel size 3, padding 1, and stride 1. Finally, a tail (T) may close the decoding path 704 to obtain the output. The tail may consist of the sequential application of group normalization, Swish function and a 2D convolutional layer with kernel size 3, stride 1 and size 1.

Turning to FIG. 1B, FIG. 1B is a flowchart illustrating an example process 110 for training a generative model used as part of the process 100 (e.g., in step 106 of the process 100) to generate the second image, hereinafter referred to as training process 110. In some examples, the generative model may be the first generative model 124 (e.g., the cGAN) used to generate the synthetic 4D CT image 126, where the cGAN could be a Variant A model or Variant B model. In other examples, the generative model may be the second generative model 125 (e.g., the cGAN or the cDDPM) used to generate the synthetic SPECT/CT image 127. Differences in training among the different model types are addressed in the description to follow.

The training process 110 may be executed by one or more machines, such as a computing device or system as described below with reference to FIG. 8. Often, the machine configured to execute the process 110 for training the generative model may be a different machine than the machine configured to execute the inference process 100 described above with reference to FIG. 1A. However, in some examples, a same machine may execute both the inference process 100 and the training process 110. The training process 110 may include one or more of the following steps.

In step 112, the training process 110 may include receiving a plurality of training datasets. When the generative model being trained is the cGAN model (e.g., both Variant A and Variant B), each training dataset may include at least a training 3D CT image, a training output of the lung simulation model generated based on the training 3D CT image, and a real 4D CT image from which the training 3D CT image is obtained (e.g., the training 3D CT image is one of multiple 3D CT image comprising the real 4D CT image). The real 4D CT image may be used as a ground truth. If the cGAN model being trained is the Variant B model, each training dataset may also include training displacement vector fields. When the generative model being trained is the cGAN (not including, for example, Variant A and Variant B) or the cDDPM, each training dataset may include at least a training 3D CT image, a training output of the lung simulation model generated based on the training 3D CT image, and a real SPECT image corresponding to training 3D CT image. The real SPECT image may used as a ground truth.

The data included in the training datasets may be collected from internal and/or external resources associated with healthcare provider systems, imaging provider systems, laboratory systems, etc. In some examples, the training data or at least certain portions of the training data may undergo preprocessing prior to providing the training data to the generative model for processing.

In step 114, the training process 110 may include generating and training a generative model using at least a portion of the plurality of training datasets. In some examples, another portion of the training datasets are withheld to test and/or validate the generative model. For example, the training 3D CT image and the training output of the lung simulation model generated based on the training 3D CT image (and the displacement vector fields if Variant B of the cGAN) may be input to generative model. The generative model may generate a synthetic image (e.g., the synthetic 4D CT image or synthetic SPECT image) and provide the synthetic image as output.

In one example, to train the generative model, the output may be compared to the ground truth (e.g., the real 4D CT Image or real SPECT image) corresponding to the 3D CT training image provided as input to determine a loss or error. The generative model may be modified or altered (e.g., weights and/or bias may be adjusted) based on the error to improve an accuracy of the machine learning system. This process may be repeated for the portion of the plurality of training datasets or at least until a determined loss or error is below a predefined threshold. As previously mentioned, some of the training datasets may be withheld and used to further validate or test the trained generative model.

In step 116, the training process 110 may comprise storing the trained generative model for subsequent deployment to perform the process 100.

### V. Benefits

The following benefits and use cases result from the generation of synthetic 4D CT or SPECT images from a single 3D CT image using one or more of the processes described above:
Replace laborious and complex image acquisition, as well as generally minimize the number of images needed to evaluate the condition of a patient. Real 4D CT and SPECT image acquisition is a laborious and complex process depending on the specific technique used to image the patient, as it takes time to perform the entire imaging technique and may require additional equipment.
Provide synthetic 4D CT or SPECT images for patients who cannot undergo the imaging procedure required to obtain real 4D CT or SPECT images due to their unstable condition. Since image acquisition may be time-consuming and might require the patient's cooperation, patients in unstable conditions cannot undergo the procedure. For other patients, it may be beneficial to avoid additional radiation doses. Synthetic 4D CT or SPECT images can provide the necessary image information for these patients.
Generate synthetic 4D CT or SPECT images for respiration maneuvers that were not imaged. Real 4D CT and SPECT images are limited to the respiration maneuver captured during imaging. Synthetic images can be generated for arbitrary breathing curves or particular points in the breathing cycle, for both respiration and ventilation.
Generate a second CT image at a different pressure level during mechanical ventilation. Recruitment/de-recruitment of small airways can be assessed with two CT images taken during at least two different breath hold maneuvers with different pressure levels. Hence, one use case for approach described herein may be prediction or quantification of recruitment/de-recruitment during mechanical ventilation without being able to account for this phenomenon in the lung simulation model. The lung simulation model would effectively provide only a proxy for the regional information, and the generative approach would transform this together with the static CT image into a hypothetical/simulated second CT image (or a sequence) which would then allow us to quantify recruitment/de-recruitment more precisely. This approach requires further processing of the CT images to quantify recruitment/de-recruitment in the respective scans. This information would the allow us to choose good/optimal settings of a ventilator, e.g., the PEEP, peak pressure, plateau pressure, tidal volume etc.
Generate SPECT/deposition fields without simulating particle transport. A strain field could be used to condition the GAN to create synthetic SPECT images or deposition maps without having to simulate particle transport and deposition. This would greatly reduce the computational costs associated with a deposition analysis and provide near instantaneous results.
Generate synthetic training data for deep learning-based image registration techniques. Machine and deep learning models require a conspicuous amount of training data, which may be difficult to obtain in a clinical setting. Moreover, their medical applications often generate concerns due to the need to protect patients' data. In this case, synthetic images can provide a solution to both problems.
Enable retrospective analysis of patients where only a single CT image is available.
Generate synthetic images of different imaging modalities. Transfer CT to MRI or SPECT. Depending on the modality different model output quantities could be used (stress, strain, perfusion, etc.).

### VI. Environment for Implementing Embodiments

In general, any process or operation discussed in this disclosure that is understood to be machine- or computer-implementable may be performed by one or more processors of a computer system. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

A computer system, such as a system or device implementing a process or operation in the examples above, may include one or more computing devices. One or more processors of a computer system may be included in a single computing device or distributed among a plurality of computing devices. A memory of the computer system may include the respective memory of each computing device of the plurality of computing devices.

FIG. 8 depicts an example of a computer 800, according to certain aspects. FIG. 8 is a simplified functional block diagram of a computer 800 that may be configured as a device for executing processes or operations depicted in, or described with respect to, FIGS. 1A-7, according to exemplary aspects of the present disclosure. In various aspects or examples, any of the systems herein may be a computer 800 including, e.g., a data communication interface 820 for packet data communication. The computer 800 may communicate with one or more other computers 800 using the electronic network 826.

The computer 800 also may include a central processing unit (“CPU”), in the form of one or more processors 802, for executing program instructions 824. The program instructions 824 may include instructions for running one or more operations of the respective device or system, such as the inference process 100 or the training process 110. The computer 800 may include an internal communication bus 806, and a drive unit 806 (such as read-only memory (ROM), hard disk drive (HDD), solid-state disk drive (SDD), etc.) that may store data on a computer readable medium 822, although the computer 800 may receive programming and data via network communications. The computer 800 may also have a memory 804 (such as random access memory (RAM)) storing instructions 824 for executing techniques presented herein, although the instructions 824 may be stored temporarily or permanently within other modules of computer 800 (e.g., processor 802 or computer readable medium 822). The computer 800 also may include user input and output ports 812 or a display 810 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, e.g., may enable loading of the software from one computer or processor into another. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While the disclosed methods, devices, and systems are described with exemplary reference to transmitting data, it should be appreciated that the disclosed aspects may be applicable to any environment, such as a desktop or laptop computer, an automobile entertainment system, a home entertainment system, etc. Also, the disclosed aspects may be applicable to any type of Internet protocol.

It should be understood that aspects in this disclosure are exemplary only, and that other aspects may include various combinations of features from other aspects, as well as additional or fewer features.

It should be appreciated that in the above description of exemplary aspects of the invention, various features of the invention are sometimes grouped together in a single aspect, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some aspects described herein include some but not other features included in other aspects, combinations of features of different aspects are meant to be within the scope of the invention, and form different aspects, as would be understood by those skilled in the art. For example, in the following claims, any of the claimed aspects can be used in any combination.

Thus, while certain aspects have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A machine-implemented method comprising:
receiving at least a first image of at least a portion of an organ, the first image obtained using a first imaging modality;
determining a computational model that simulates and/or predicts, using the first image, one or more functional, mechanical, chemical, biological, and/or physiological quantities across a domain of at least the portion of the organ; and
providing an output of the computational model to a generative model, the generative model comprising one or more of generative adversarial network(s) (GANs), diffusion model(s), and/or other generative model(s) to:
receive and/or generate a second image of the organ obtained using a second imaging modality different from the first imaging modality,
generate a second image of the organ associated with the first imaging modality under different real or simulated conditions than the first image, the different real or simulated conditions comprising different time, different mechanical, or different physiological conditions, and/or
generate a combination of both.

2. The machine-implemented method according to claim 1, wherein at least the portion of the organ comprises a lung and/or respiratory system.

3. The machine-implemented method according to claim 1, wherein the output of the computational model is used to condition a generative model that was trained on a number of samples, such that the samples include data in which either the second image taken with the second imaging modality is available or the organ was imaged with the first imaging modality at another time or under other conditions.

4. The machine-implemented method according to claim 1, wherein the computational model is created and not only informed by the first image.

5. The machine-implemented method according to claim 1, wherein the first imaging modality is CT or MRI.

6. The machine-implemented method according to claim 1, wherein the second imaging modality is SPECT, CT, or MRI.

7. The machine-implemented method according to claim 1, wherein the different real or simulated conditions are different points in time, including weeks or months apart, or different breathing cycle states, such as end inspiratory or end expiratory.

8. The machine-implemented method according to claim 1, wherein the output is a strain or deposition field obtained from the computational model.

9. The machine-implemented method according to claim 1, wherein the generative model is a GAN or diffusion model.

10. The machine-implemented method according to claim 1, wherein the first imaging modality is CT, wherein at least the portion of the organ comprises a lung, wherein the output is a deposition field obtained from the computational model, wherein the deposition field is used to generate the second image, and wherein the second imaging modality is SPECT.

11. The machine-implemented method according to claim 1, wherein the first imaging modality is CT, wherein at least the portion of the organ comprises a lung, wherein the output is a strain field obtained from the computational model, wherein the strain field is used to generate the second image, wherein the first image is a first CT image at a first time of a breathing cycle, and wherein the second image is a second CT image at a second time of the breathing cycle different than the first time.

12. The machine-implemented method according to claim 11, further comprising determining recruitment/de-recruitment of at least one airway based on the first CT image and the second CT image.

13. The machine-implemented method according to claim 11, wherein the first CT image and the second CT image are used to determine a ventilator setting for a patient.

14. A system comprising:
a medical imaging device; and
a computer system in communication with the medical imaging device, the computer system including at least one memory component and at least one processor component, the at least one processor component being configured to perform operations comprising:
receiving at least a first image of at least a portion of an organ, the first image obtained using a first imaging modality;
determining a computational model that simulates and/or predicts, using the first image, one or more functional, mechanical, chemical, biological, and/or physiological quantities across a domain of at least the portion of the organ; and
providing an output of the computational model to a generative model, the generative model comprising one or more of generative adversarial network(s) (GANs), diffusion model(s), and/or other generative model(s) to:
receive and/or generate a second image of the organ obtained using a second imaging modality different from the first imaging modality,
generate a second image of the organ associated with the first imaging modality under different real or simulated conditions than the first image, the different real or simulated conditions comprising different time, different mechanical, or different physiological conditions, and/or
generate a combination of both.

15. A method comprising:
capturing a first medical image of a respiratory system via a first imaging modality;
generating a computational model based on the first medical image,
wherein the computational model is configured to simulate or predict at least one of functional, mechanical, chemical, biological, or physiological quantities of the respiratory system based on the first medical image; and
executing a generative model on an output of the computational model to generate a second medical image of the respiratory system.

16. The method of claim 15, wherein executing the generative model comprises generating the second medical image using a second imaging modality different from the first imaging modality.

17. The method of claim 16, wherein the first imaging modality is CT and wherein the second imaging modality is SPECT.

18. The method of claim 15, wherein executing the generative model comprises generating the second medical image under different mechanical or physiological conditions of the first imaging modality.

19. The method of claim 15, wherein the generative model includes at least one of generative adversarial networks (GANs) or diffusion models.

20. The method of claim 15, wherein the first medical image is a first CT image that is captured at a first time in a breathing cycle of the respiratory system, and wherein the second medical image is a second CT image that simulates a second time in the breathing cycle different than the first time.

21. The method of claim 15, further comprises determining at least one ventilator setting based on the first medical image and the second medical image.

22. The method of claim 15, wherein the output of the computational model is a deposition field, and wherein executing the generative model comprises inputting the deposition field to generate the second medical image.

23. The method of claim 22, wherein executing the generative model comprises generating the second medical image using a second imaging modality, wherein the second imaging modality is SPECT.

24. The method of claim 15, wherein the output of the computational model is a strain field, and wherein executing the generative model comprises inputting the strain field to generate the second medical image.

25. The method of claim 23, wherein the first medical image is a first CT image that is captured at a first time in a breathing cycle of the respiratory system, and wherein the second medical image is a second CT image that simulates a second time in the breathing cycle different than the first time.

26. A system comprising:
a medical imaging device; and
a computer system in communication with the medical imaging device, the computer system including at least one memory component and at least one processor component, the at least one processor component being configured to perform operations comprising:
capturing a first medical image of a respiratory system via a first imaging modality of the medical imaging device;
generating a computational model based on the first medical image, wherein the computational model is configured to simulate or predict at least one of functional, mechanical, chemical, biological, or physiological quantities of the respiratory system based on the first medical image; and
executing a generative model on an output of the computational model to generate a second medical image of the respiratory system.
